(19) United States
(12) Patent Application Publication
(10) Pub. No.: US 2003/0044780 A1
(43) Pub. Date: Mar. 6, 2003

(54) PRIMER EXTENSION METHODS UTILIZING DONOR AND ACCEPTOR MOLECULES FOR DETECTING NUCLEIC ACIDS

(76) Inventors: STANLEY N. LAPIDUS, BEDFORD, NH (US); ANTHONY P. SHUBER, MILFORD, MA (US)

Correspondence Address: TESTA, HURWITZ & THIBEAULT, LLP, HIGH STREET TOWER, 125 HIGH STREET, BOSTON, MA, 02110, US

(21) Appl. No.: 09/448,042

(22) Filed: Nov. 23, 1999

Related U.S. Application Data
(60) Provisional application No. 60/109,599, filed on Nov. 23, 1998.

(51) Int. Cl. C12Q 1/68; C12P 19/34

(52) U.S. Cl. 435/6; 435/91.2

(57) ABSTRACT

Methods are provided for selective nucleic acid sequence detection in primer extension reactions of high specificity. These methods are useful for detecting small amounts of mutant nucleic acid in a heterogeneous biological sample. These methods are particularly useful for identifying individuals with gene mutations indicative of early colorectal cancer.

### FIELD OF THE INVENTION

The present invention relates generally to methods for identifying a nucleic acid present in a biological sample. Methods of the invention are useful for disease diagnosis by detecting genetic mutations and identifying low-frequency molecular events.

### BACKGROUND OF THE INVENTION

Numerous methods have been devised to detect the presence of genetic mutations. A variety of detection methods have been developed which exploit sequence variations in DNA using enzymatic and chemical cleavage techniques. A commonly-used screen for DNA polymorphisms consists of digesting DNA with restriction endonucleases and analyzing the resulting fragments by means of southern blots, as reported by Botstein et al., Am. J. Hum. Genet., 32:314-331 (1980) and White et al., Sci. Am., 258:40-48 (1988). Mutations that affect the recognition sequence of the endonuclease will preclude enzymatic cleavage at that site, thereby altering the cleavage pattern of the DNA. Sequences are compared by looking for differences in restriction fragment lengths. A problem with this method (known as restriction fragment length polymorphism mapping or RFLP mapping) is its inability to detect mutations that do not affect cleavage with a restriction endonuclease. One study reported that only 0.7% of the mutational variants estimated to be present in a 40,000 base pair region of human DNA were detected using RFLP analysis. Jeffreys, Cell, 18:1-18 (1979).

In enzyme-mediated ligation methods, a mutation is interrogated by two oligonucleotides capable of annealing immediately adjacent to each other on a target DNA or RNA molecule, one of the oligonucleotides having its 3′ end complementary to the point mutation. Adjacent oligonucleotides are only covalently attached when both oligonucleotides are correctly base-paired. Thus, the presence of a point mutation is indicated by the ligation of the two adjacent oligonucleotides. Grossman et al., Nucleic Acid Research, 22:4527-4534 (1994). However, the usefulness of this method for detection is compromised by high backgrounds which arise from tolerance of certain nucleotide mismatches or from non-template directed ligation reactions. Barringer et al., Gene, 89:117-122 (1990).

Single base mutations have also been detected by differential hybridization techniques using allele-specific oligonucleotide (ASO) probes. Saiki et al., Proc. Natl. Acad. Sci. USA, 86:6230-6234 (1989). Mutations are identified on the basis of the higher thermal stability of the perfectly-matched probes as compared to mismatched probes. That approach to mutation analysis requires optimization of hybridization for each probe, and the nature of the mismatch and the local sequence impose limitations on the degree of discrimination of the probes. In practice, tests based only on parameters of nucleic acid hybridization function poorly when the sequence complexity of the test sample is high (e.g., in a heterogeneous biological sample). This is partly due to the small thermodynamic differences in hybrid stability generated by single nucleotide changes.

A number of detection methods have been developed which are based on a template-dependent, primer extension reaction. These methods fall essentially into two categories: (1) methods using primers which span the region to be interrogated for the mutation, and (2) methods using primers which hybridize proximally of the region to be interrogated for the mutation.

In the first category, Caskey and Gibbs, U.S. Pat. No. 5,578,458, report a method wherein single base mutations in target nucleic acids are detected by competitive oligonucleotide priming under hybridization conditions that favor the binding of the perfectly-matched primer as compared to one with a mismatch. Vary and Diamond, U.S. Pat. No.4,851,331, reported a similar method wherein the 3′ terminal nucleotide of the primer corresponds to the variant nucleotide of interest. Since mismatching of the primer and the template at the 3′ terminal nucleotide of the primer inhibits elongation, significant differences in the amount of incorporation of a tracer nucleotide result under normal primer extension conditions.

It has long been known that primer-dependent DNA polymerases have, in general, a low replication error rate. This feature is essential for the prevention of genetic mistakes which would have detrimental effects on progeny. Methods in a second category exploit the high fidelity inherent in this enzymological reaction. Detection of mutations is based on primer extension and incorporation of detectable, chain-terminating nucleotide triphosphates. The high fidelity of DNA polymerases ensures specific incorporation of the correct base labeled with a reporter molecule. Such single nucleotide primer-guided extension assays have been used to detect aspartylglucosaminuria, hemophilia B, and cystic fibrosis; and for quantifying point mutations associated with Leber Hereditary Optic Neuropathy (LHON). See. e.g., Kuppuswamy etal., Proc. Natl. Acad. Sci. USA, 88:1143-1147 (1991); Syvanen etal., Genomics, 8:684-692 (1990); Juvonen et al., Human Genetics, 93:16-20 (1994); Ikonen et al., PCR Meth. Applications, 1:234-240 (1992); Ikonen et al., Proc. Natl. Acad. Sci. USA, 88:11222-11226 (1991); Nikiforov et al., Nucleic Acids Research, 22: 4167-4175 (1994). An alternative primer extension method involving the addition of several nucleotides prior to the chain-terminating nucleotide has also been proposed in order to enhance resolution of the extended primers based on their molecular weights. See, e.g., Fahy et al., WO/96/30545 (1996).

The selectivity and specificity of an oligonucleotide primer extension assay are related to the length of the oligonucleotide primer, and to the reaction conditions. In general, primer lengths and reaction conditions that favor high selectivity result in low specificity. Conversely, primer lengths and reaction conditions that favor high specificity result in low selectivity.

There is a need in the art for a single base extension assay that has greater selectivity and specificity than available methods. Such methods are provided herein.

### SUMMARY OF THE INVENTION

The invention provides methods of detecting and identifying low-frequency molecular events (e.g., nucleic acid mutations). Methods of the invention comprise a single base extension assay in which donor and acceptor molecules are positioned in proximity to each other upon the addition of a single nucleotide to a primer. In a preferred embodiment, a primer comprising a donor molecule is annealed immediately upstream of a single base to be interrogated. In the presence of a polymerase, a nucleotide comprising an acceptor molecule capable of interacting with the donor molecule to produce a detectable signal is extended on the primer. Alternatively, the donor molecule is associated with the added nucleotide, and the acceptor molecule is associated with the primer. Proximity of the donor and acceptor molecules produces a signal that is associated with the added nucleotide, and therefore identifies its complement on the target.

Methods of the invention are useful for detecting mutant DNA that exists in heterogeneous biological samples, such as stool, sputum, or pap smears. In such samples, the informative DNA (i.e., clinically relevant mutant DNA) available for analysis is limited by the presence of large amounts of non-relevant DNA, and by the small amounts of relevant DNA in the sample. Accordingly, methods of the invention provide means for maintaining or improving the ratio of mutant DNA to wild-type DNA in the sample. In preferred embodiments, a sufficient number of molecules is presented to an amplification reaction (e.g., PCR) in order to promote amplification and detection of the mutant. Also in a preferred embodiment, the ratio of labeled mutant DNA is proportional to the amount of wild-type DNA that is labeled, thus avoiding contamination of the mutant signal.

Primers for use in methods of the invention may be an oligo- or poly-nucleotides having a donor molecule attached thereto or incorporated therein, and which are complementary to a portion of target nucleic acid immediately upstream (i.e., with one or no intervening target bases) to the single base target to be interrogated. Preferred primer lengths are those recognized by a DNA polymerase. A preferred primer length, therefore, is greater than about 8 nucleotides. The primer can be any length that is practical in the context of the assay to be conducted.

Nucleotides for use in the methods of the invention may be any nucleotide having an acceptor molecule attached thereto or incorporated therein, and which is complementary to the single base target. In a preferred embodiment, nucleotides used in the invention are chain-terminating nucleotides, for example dideoxynucleotides ddATP, ddCTP, ddGTP, and ddTTP.

Donor and acceptor molecules for use in the methods of the invention may comprise fluorophore. In a preferred embodiment, the donor and acceptor molecules comprise a fluorescent dye selected from the group consisting of CyDye™ (Amersham), 6-carboxyfluorescein (FAM, Amersham), 6-carboxy-X-rhodamine (REG, Amersham), N₁, N₁ N¹, N¹-tetramethyl-6-carboxyrhodamine (TAMARA, Amersham), 6-carboxy-X-rhodomine (ROX, Amersham), flourescein, Cy5® (Amersham) or LightCycler-Red 640 (Roche Molecular BioChemicals). In a more preferred embodiment, the donor molecules comprise FAM and the acceptor molecules comprise REG, TAMARA or ROX. In an alternate embodiment, the donor molecules comprise flourescein, and the acceptor molecules comprise Cy5® (Amersham) or LightCycler-Red 640 (Roche Molecular BioChemicals).

In a preferred embodiment, a plurality of chain-terminating nucleotides, comprising different (although not mutually exclusive) acceptor molecules is provided. The use of a plurality of nucleotides comprising acceptor molecules allows identification of the relative amounts of alternative nucleotides at the position in the target that is complementary to the extended base. This allows, for example, analysis of single nucleotide polymorphic variants.

In a preferred embodiment, methods of the invention are used to determine the relative amounts of nucleotides present at a heterozygous polymorphic locus. Such methods are useful, for example, to determine whether a loss of heterozygosity has occurred at the locus. In a normal (i.e., healthy) individual who is heterozygous at a polymorphic locus, the number or amount of one allele (e.g., the maternal allele) should be equal to the number or amount of the other allele (e.g., the paternal allele). If the numbers/amounts of the two allele (as determined by the numbers/amounts of the two single nucleotide variants at the heterozygotic locus) are different (by a statistically-significant amount), there is evidence of a loss of heterozygosity or an increase in the number of one of the interrogated alleles. Amounts may be determined by standard bulk detection methods or by enumeration. Methods for enumerating single nucleotides to detect LOH are taught in U.S. Pat. No. 5,624,325, incorporated by reference herein.

In a preferred embodiment, the present invention also provides methods for detecting polymorphic changes in a subpopulation of cells in a pooled sample obtained by collecting samples from members of a patient population (e.g. healthy, diseased, heterozygotes, etc.). Methods of the invention are useful for the detection of changes in the nucleotide sequence of an allele in a small subpopulation of cells present in a large, heterogeneous sample of diagnostically-irrelevant biological material. Practice of the invention permits, for example, detection of trace amounts of DNA derived from cancer or precancer cells in pooled biological samples.

Methods disclosed herein may be used to detect mutations associated with genetic, infectious and parasitic diseases. In a preferred embodiment, the methods recited herein may be used to detect cancer. Other non-limiting examples of detectable diseases include cystic fibrosis, Tay-Sachs disease, sickle-cell anemia, ∝ and β-thalassemia, phenlyketonuria, hemophilia, α-anti-trypsin deficiency, Gaucher's disease, insulin-resistant diabetes, HIV, and hepatitis.

Methods disclosed herein may be used to detect mutations in genes associated with cancer such as, the ras oncogenes, p53, dcc, apc, mcc and β-catenin. Other genes associated with cancer are well known in the art. See e.g., Hesketh R., The Oncogene Facts Book, Academic Press, 1988, incorporated by reference herein.

Methods of the invention may be performed on any biological sample, including tissue and body fluid samples. Particularly preferred biological samples include stool, pus, sputum, semen, blood, saliva, cerebrospinal fluid, urine, biopsy tissue and lymph.

Further aspects and advantages of the invention are apparent upon consideration of the following detailed description thereof.

### DESCRIPTION OF THE DRAWINGS

FIG. 1 is a diagram showing sequential steps in the method for identifying a nucleic acid as exemplified below.

### DETAILED DESCRIPTION OF THE INVENTION

The invention comprises single base extension assays that detect low-frequency molecular events in a biological sample. The present invention provides methods for detecting specific nucleic acids in a biological sample with both high sensitivity and high specificity. In general, methods of the invention comprise performing a single-base extension reaction utilizing donor and acceptor molecules which interact to produce a detectable signal. Methods of the invention are useful to detect and identify mutations associated with diseases such as cancer. Methods of the invention are also useful to detect deletions or base substitutions, such as those causative of a metabolic error, such as complete or partial loss of enzyme activity. Methods of the invention are useful to identify and assay single nucleotide polymorphisms (SNPs). For purposes of the present invention, unless the context requires otherwise, a “mutation” includes modifications, rearrangements, deletions, substitutions and additions in a portion of genomic DNA or its corresponding RNA.

Methods of the invention comprise a single base extension assay in which a primer for extension comprises a donor molecule and anneals to its complementary DNA in a sample. For exemplification, the single base extension assay is shown with DNA however the assay could also be performed with cDNA or RNA. In the presence of a polymerase, the primer is extended one base by a nucleotide comprising an acceptor molecule. When the primer is extended, the donor and acceptor molecules are in proximity to interact. The interaction of the donor and acceptor molecules produces a detectable signal which is uniquely associated with the added nucleotide, and therefore identifies its complement on the target.

For convenience, the primer is characterized as comprising a donor molecule and the nucleotide is characterized as comprising an acceptor molecule. The skilled artisan will appreciate that the location of the donor and acceptor could be reversed. Namely, the primer could comprise an acceptor molecule and the nucleotide could comprise a donor molecule.

Methods of the invention further comprise conducting multiple cycles of a single-base extension reaction in a biological sample. By cycling, extended product yield is high, and there is no significant loss of specificity because hybridization conditions for the primer are kept stringent relative to those typically applied during a single-base extension reaction. Further details regarding the cycling of a single base extension reaction is disclosed in copending patent application Ser. No. 08/067,212 (attorney docket No. EXT-016), which is incorporated by reference herein.

I. Primer Extension Reaction Utilizing Donor and Acceptor Molecules

A primer extension reaction is performed by exposing DNA isolated from a biological sample and optionally amplified using PCR to a nucleic acid primer that is complementary to a portion of the DNA. Once annealed, the 3′ end of the primer is extended by one base, using a nucleotide comprising an acceptor molecule, in a template-directed reaction catalyzed by a polymerase. The nucleic acid primer includes a donor molecule which is capable of interacting with the acceptor molecule on the extended base (once the base is incorporated in the primer) to produce a detectable signal. The signal is uniquely-associated with the extended base, and therefore its complement on the target.

A primer, comprising a donor molecule preferably is designed so that the hybridized primer is immediately upstream of the position that is complementary to the nucleotide position being assayed. The primer extension reaction is performed in the presence of a nucleotide, preferably a chain-terminating nucleotide, comprising an acceptor molecule and a DNA polymerase. The polymerase adds a nucleotide comprising an acceptor molecule to the end of the primer. The nucleotide position being assayed is identified as the nucleotide that is complementary to the nucleotide incorporated in the single-base primer extension reaction. When the nucleotide is incorporated into the primer, the donor molecule causes the acceptor to produce a detectable signal that is measured and quantified. The donor and acceptor molecules interact to produce a detectable signal characteristic of the interaction only when they are in close proximity. The donor and acceptor must be close enough to allow for sufficient interaction (i.e., energy transfer) but far enough apart to avoid self-quenching. In a preferred embodiment the signal produced by the acceptor is a photo-emitting signal.

In a preferred embodiment, the single-base extension reaction utilizes segmented primers. A segmented primer comprises at least two oligonucleotide probes, a first probe and a second probe, which are capable hybridizing to substantially contiguous portions of a nucleic acid. Either the first or second probe may comprise a donor molecule. Generally, the shorter probe comprises the donor molecule. Neither probe alone is capable of being a primer for template-dependent extension, but when the probes hybridize adjacent to each, they are capable of priming extension. In a preferred embodiment, methods of the invention comprise hybridizing to a target nucleic acid a probe having a length from about 5 bases to about 10 bases, wherein the probe hybridizes immediately upstream of a single nucleotide locus to be interrogated. A second probe is hybridized upstream of the first probe and having a length from about 15 to about 100 nucleotides and a 3′ non-extendible nucleotide. Preferably, substantially contiguous probes are between 0 and 1 nucleotides apart. Further details regarding the use of segmented primers is disclosed in copending patent application Ser. No. 08/877,333 (attorney docket No. EXT-007), now allowed, which is incorporated by reference herein.

In a preferred embodiment, the extension reaction is performed in the presence of numerous chain-terminating nucleotides comprising different acceptor molecules which produce distinct signals. In an alternate embodiment, less than all the chain-terminating nucleotides comprise an acceptor molecule. If the biological sample is heterogeneous at the nucleotide position being assayed, the complementary nucleotides (if they are included in the primer extension reaction) will be incorporated in the primer extension assay.

In a preferred embodiment, methods according to the invention also may be used to detect a loss of heterozygosity at an allele by determination of the number of or amounts of maternal and paternal alleles comprising a genetic locus that includes at least one single-base polymorphism. A statistically-significant difference in the numbers or amounts of each allele is indicative of a mutation in an allergic region encompassing the single-base polymorphism. In this method, a region of an allele comprising a single-base polymorphism is identified, using, for example, a database, such as GenBank, or by other means known in the art. Probes are designed to hybridize to corresponding regions on both paternal and maternal alleles immediately 3′ to the single base polymorphism. After hybridization, a mixture of at least two chain terminating nucleotides are added to the sample, each comprising distinct acceptor molecules. A DNA polymerase is also added. Using allelic DNA adjacent the polymorphic nucleotide as a template, hybridized probe is extended by the addition of a single nucleotide that is the binding partner for the polymorphic nucleotide. The chain-terminating nucleotides which have been incorporated into the primer are detected by determining the number of or amount of bound extended probes bearing each of the two chain-terminating nucleotides. The presence of an equal number or amount of two different acceptors within predefined statistical limits, as described in U.S. Pat. No. 5,670,325, which is incorporated by reference, mean that there is normal heterozygosity at the polymorphic nucleotide. The presence of a statistically-significant difference between the detected numbers of or amounts of the two acceptors means that a deletion of the region encompassing the polymorphic nucleotide has occurred in one of the alleles.

II. Detection of Extended Primers

The nucleotides comprise an acceptor molecule which interacts with a donor molecule on the primer when in close proximily and thus facilitates detection of the extended primers, or extended short first probes in an extension reaction. The donor and acceptor molecules may comprise a fluorophore. In preferred embodiments, the donor and acceptor molecules comprise a fluorescent dye such 6-carboxyfluorescein (FAM, Amersham), 6-carboxy-X-rhodamine (REG, Amersham), N₁, N₁ N¹, N¹-tetramethyl-6-carboxyrhodamine (TAMARA, Amersham), 6-carboxy-X-rhodomine (ROX, Amersham), fluorescein, Cy5® (Amersham) and LightCycler-Red 640 (Roche Molecular Biochemicals). In a preferred embodiment, the donor molecules comprise FAM and the acceptor molecules comprise REG, TAMARA or ROX. In an alternate embodiment the donor is fluoroscein and the acceptor is Cy5® or LightCycler-Red 640 (Roche Molecular Biochemicals). Alternatively, the donor and acceptor molecules comprise fluorescent labels such as the dansyl group, substituted fluorescein derivatives, acridine derivatives, coumarin derivatives, pthalocyanines, tetramethylrhodamine, Texas Red ®, 9-(carboxyethyl)-3-hydroxy-6-oxo-6H-xanthenes, DABCYL®, BODIPY® (Molecular Probes, Eugene, Oreg.) can be utilized. Such labels are routinely used with automated instrumentation for simultaneous high throughput analysis of multiple samples.

Fluorescence monitoring of amplification is based on the concept that a fluorescence resonance energy transfer occurs between two adjacent fluorophores and a measurable signal is produced. When an external light source, such as a laser or lamp-based system is applied, the donor molecule is excited and it emits light of a wavelength that in turn excites an acceptor molecule that is in close proximity to the donor molecule. The acceptor molecule then emits an identifiable signal (i.e., a fluorescent emission at a distinct wavelength) that can measured and quantified. The donor molecule does not transmit a signal to acceptor molecules that are not in close proximity. Thus, when the ddNTP incorporates into the primer, the donor and acceptor molecules are brought close together and a fluorescence energy transfer occurs between the two fluorophores causing the acceptor molecule to emit a detectable signal. Acceptor molecules that are in close proximity to donor molecule emit a signal that is distinctly different from the acceptor molecules alone (i.e., an acceptor molecule that is not in proximity with the donor). In addition, multiple different acceptor molecules may be used, in which each acceptor “combines” with the same donor molecule to produce distinct signals, each being characteristic of a specific donor-acceptor combination. Monitoring the fluorescence emission from the acceptor fluorophore after excitation of the donor fluorophore allows highly sensitive product quantification and genotyping.

The following examples provide details of methods according to the invention. For purposes of exemplification, the following examples provide details of the use of the methods of the present invention in colon cancer detection. Accordingly, while exemplified in the following manner, the invention is not so limited and the skilled artisan will appreciate its wide range of applicability upon consideration thereof.

### EXAMPLES

I. Exemplary Method for Detection of Colon Cancer or Precancer

A. Summary

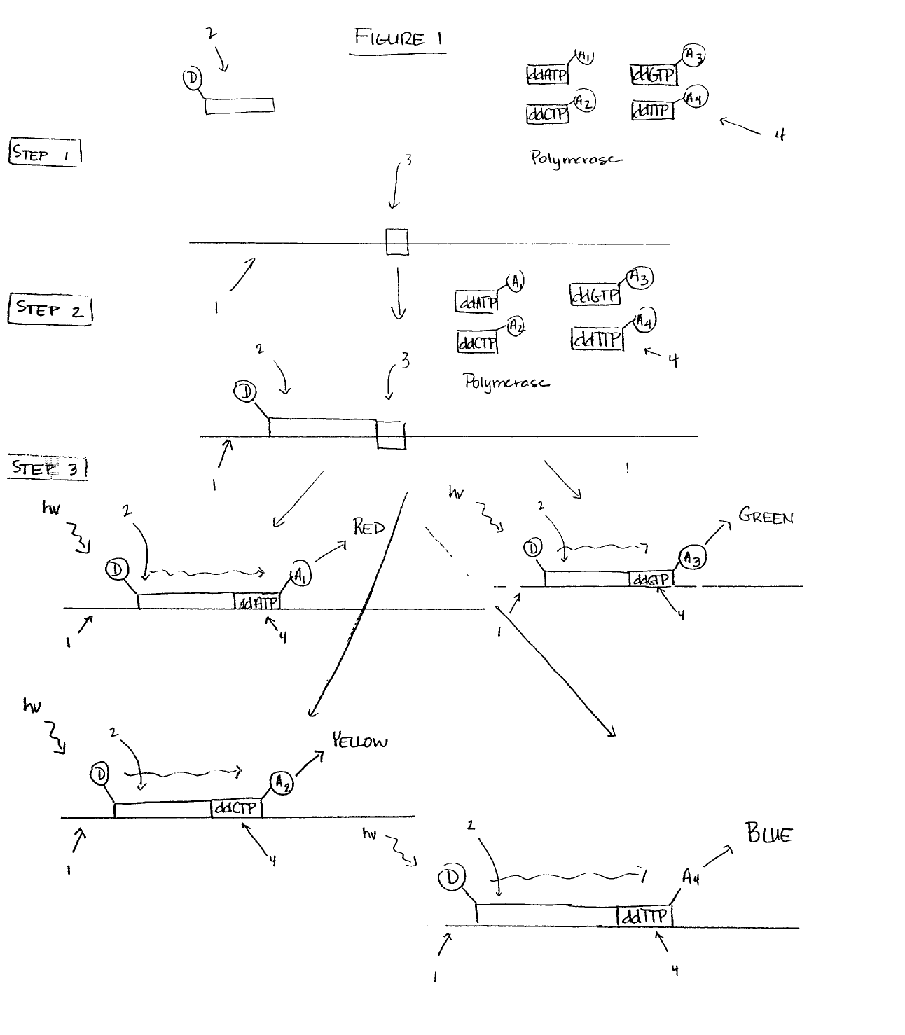

A representative stool sample is prepared as described below. Single-stranded DNA is prepared and is optionally amplified using PCR. Under conditions that promote hybridization, a single base extension reaction is conducted. The single-stranded DNA 1 is exposed to primers comprising donor molecules 2, ddNTPs (ddATP, ddCTP, ddGTP, ddTTP) 4 having different acceptor molecules and polymerase (FIG. 1, step 1). The primers are designed on the basis of a known single base polymorphism in the interrogated allele, and are prepared as described below. A primer comprising a donor 2 hybridizes with a desired number of nucleotides upstream of the polymorphic site 3 (FIG. 1, step 2). The polymerase then adds a ddNTP 4 comprising an acceptor molecules to the end of each primer, the incorporated nucleotide being complementary to the nucleotide being assayed (FIG. 1, step 3). After hybridization is complete, the sample is optionally washed to remove unbound primer and ddNTPs. When in close proximity the donor and acceptor interact to produce a photo-emitting signal that is uniquely associated with the added nucleotide and therefore identifies it complement on the target. The signal can be measured and quantified.

B. Sample Preparation of Stool

A sample is prepared that contains a representative typically, a (cross-sectional or circumferential) portion of stool. Preferred methods for preparing a representative stool sample are provided in co-owned U.S. Pat. No. 5,741,650, and in co-owned co-pending patent application Ser. No. 09/059,713 (Attorney docket No. EXT-01 5), each of which is incorporated by reference herein. As stool passes through the colon, it adheres cells and cellular debris sloughed from colonic epithelial cells. Similarly, cells and cellular debris are sloughed by a colonic polyp (comprising mutated DNA). However, only the portion of stool making contact with the polyp will adhere sloughed cells. It is therefore necessary to obtain at least a cross-sectional or circumferential portion of stool in order to ensure that the stool sample contains a mixture of all sloughed cells, including those sloughed by presumptive cancer cells (e.g., polyps).

After sample preparation, the sample is homogenized in an appropriate buffer, such as phosphate buffered saline comprising a salt, such as 20-100 mM NaCl or KCl, and optionally a detergent, such as 1-10% SDS or Triton™, and/or a proteinase, such as proteinase K. An especially-preferred buffer is a Tris-EDTA-NaCl buffer in a solvent volume to stool mass ratio of 20:1 (volume:volume) as disclosed in co-owned, co-pending U.S. patent application Ser. No. 08/876,638, [Attorney Docket No.: EXT-006], incorporated by reference herein. Homogenization means and materials for homogenization are generally known in the art. See e.g. U.S. Pat. No. 4,202,279, incorporated by reference herein. Methods for further processing and analysis of a biological sample, such as a stool sample is provided below.

DNA or RNA may be optionally isolated from the sample according to methods know in the art. See, Smith-Ravin et. al., Gut, 36:81-86, incorporated by reference herein. However, methods of the invention can be performed on unprocessed stool.

C. Preparation of Primers

Genomic regions suspected to contain one or more mutations are identified, for example, by reference to a nucleotide database, such as GenBank, EMBL, or any other appropriate database or publication, or by sequencing. For cancer detection, genetic mutations in a number of oncogenes and tumor suppressor genes are known. Duffy, Clin. Chem., 41:1410-1413 (1993). Preferred genes for use in mutation detection methods of the invention include one or more oncogenes and/or one or more tumor suppressor genes. Specifically preferred genes include the ras oncogenes, p53, dcc, apc, mcc, and other genes suspected to be involved in the development of an oncogenic phenotype.

As will be described below, methods of the invention permit the detection of a mutation at a locus in which there is more than one nucleotide to be interrogated. Moreover, methods of the invention may be used to interrogate a locus in which more than one single base mutation is possible. Once regions of interest are identified, at least one primer comprising a donor molecule is prepared to detect the presence of a suspected mutation. A primer of the invention preferably has a length from about 10 to about 100 nucleotides, more preferably between about 15 and about 35 nucleotides, and most preferably about 25 nucleotides.

The primer may be natural or synthetic, and may be synthesized enzymatically in vivo, enzymatically in vitro, or non-enzymatically in vitro. Primers for use in methods of the invention are preferably selected from oligodeoxyribonucleotides, oligoribonucleotides, copolymers of deoxyribonucleotides and ribonucleotides, peptide nucleic acids (PNAs), and other functional analogues. Peptide nucleic acids are well-known. See Pluskal, et al., The FASEB Journal, Poster #35 (1994).

For exemplification, a primer designed to detect a mutation in the K-ras gene is provided below. According to methods of the invention, primers complementary to either portions of the coding strand or to portions of the non-coding strand may be used. For illustration, a primer useful for detection of mutations in the coding strand are provided below. Mutations in K-ras frequently occur in the codon for amino acid 12 of the expressed protein.

The wild-type codon 12 of the K-ras gene and its upstream nucleotides are:

wild-type template 3′-TATTTGMCACCATCMCCTCGACCA-5′ (SEQ ID NO:1)

The three nucleotides encoding amino acid 12 are underlined. A primer (Primer 1) comprising a donor molecule (N), and capable of interrogating the first nucleotide position in the codon encoding amino acid 12 of the K-ras gene is provided below. For exemplification, the donor is shown at the 5′ end of the primer, however, it can be attached or located anywhere on the primer.

Primer 1 N-5′-ATAAACTTGTGGTAGTTGGAGCT-3′ (SEQ ID NO: 9)

In a preferred embodiment, multiple cycles of the single base extension reaction are performed, thereby increasing the specificity of the primer extension reaction without comprising selectivity. Primer 1 is hybridized to a nucleic acid sample under conditions (see Tables 1 and 2) that promote selective binding of Primer 1 to the complementary sequence in the K-ras gene. The extension reaction is performed in the presence of the 4 different dideoxynucleotides ddATP, ddCTP, ddGTP, and ddTTP, comprising distinct acceptor molecules. The extension reaction is cycled 30 times as indicated in Table 2.

TABLE 1
Reaction mixture for a single base extension cycling reaction

| Component | Amount |
|---|---|
| H2O | 25.5 |
| 10X seq Buffer | 4 |
| ddNTP (50 uM) | 5 |
| Primer (5 uM) | 5 |
| Thermo Sequenase | 0.5 |
| DNA sample | 10 |

TABLE 2
Temperature profile for a cycled single base extension reaction

| Step | Temp. (C.) | Time (Sec.) |
|---|---|---|
| 1 | 94 | 5 |
| 2 | 94 | 30 |
| 3 | 64 | 10 |
| 4 | 72 | 10 |
| 5 | Goto step 2, 29 times | |
| 6 | 4 | hold |

The reaction products are assayed for the incorporation of ddNTPs. A nucleic acid sample containing wild-type DNA should only have labeled ddGTP incorporated. The incorporation of any other ddNTP in a statistically significant amount is indicative of the presence of a mutant K-ras nucleic acid in the sample.

D. Preparation of Segmented Primers

A segmented primer comprises at least two oligonucleotide probes, a first probe and a second probe, which are capable hybridizing to substantially contiguous portions of a nucleic acid. Either the first or second probe may comprise a donor molecule. For purposes of illustration, it is assumed below that the shorter probe in the segmented pair comprises the donor molecule, unless stated otherwise.

A first probe of the invention preferably has a length of from about 5 to about 10 nucleotides, more preferably between about 6 and about 8 nucleotides, and most preferable about 8 nucleotides. A second probe of the invention has a preferable length of between about 15 and 100 nucleotides, more preferably between about 15 and 30 nucleotides, and most preferably about 20 nucleotides. Further, a second probe is incapable of being a primer for template-dependent nucleic acid synthesis absent a first probe because it has a 3′ terminal nucleotide that is non-extendible. Preferred non-extendible 3′ terminal nucleotides include dideoxy nucleotides, C3 spacers, a 3′ inverted base, biotin, or a modified nucleotide. Although, longer probes have a lower selectivity because of their tolerance of nucleotide mismatches, second probes are non-extendible and will not produce false priming in the absence of the proximal probe.

In an alternative embodiment, a segmented primer comprises a series of first probes, wherein each member of the series has a length of from about 5 to about 10 nucleotides, and most preferable about 6 to about 8 nucleotides. Although the first probes do not have a terminal nucleotide, nucleic acid extension will not occur unless all members of the series are hybridized to substantially contiguous portions of a nucleic acid.

The oligonucleotide probes of the segmented primer may be natural or synthetic, and may be synthesized enzymatically in vivo, enzymatically in vitro, or non-enzymatically in vitro. Probes for use in methods of the invention are preferably selected from oligodeoxyribonucleotides, oligoribonucleotides, copolymers of deoxyribonucleotides and ribonucleotides, peptide nucleic acids (PNAs), and other functional analogues. Peptide nucleic acids are well-known. See Pluskal, et al., The FASEB Journal, Poster #35 (1994).

For exemplification, segmented primers designed to detect mutations in the K-ras gene are provided below. According to methods of the invention, probes complementary to either portions of the coding strand or to portions of the non-coding strand may be used. For illustration, probes useful for detection of mutations in the coding strand are provided below. Mutations in K-ras frequently occur in the codon for amino acid 12 of the expressed protein. Several of the possible probes for detection of mutations at each of the three positions in codon 12 are shown below.

The wild-type codon 12 of the K-ras gene and its upstream nucleotides are:

wild-type template 3′-TATTTGAACACCATCAACCTCGACCA-5′ (SEQ ID NO: 1)

The three nucleotides encoding amino acid 12 are underlined. First probes and second probes capable of interrogating the three nucleotides coding for amino acid 12 of the K-ras gene are provided below. First probe A is a first probe as described generally above, and has a sequence complementary to the nucleotides immediately upstream of the first base in codon 12 (i.e., immediately adjacent to the cytosine at codon position 1), and comprises a donor molecule. Second probe A is a second probe as generally described above. It is complementary to a sequence that is substantially contiguous (here, exactly contiguous) with the sequence to which the first probe A is complementary. The bolded nucleotide in each of the second probes shown below is the nonextendible 3′ terminal nucleotide. Hybridization of first and second probes suitable for detection of a mutation in the first base of K-ras codon 12 are shown below:

| | | |
|---|---|---|
| second probe A | 5′-ATAAACTTGTGGTAG | (SEQ ID NO:2) |
| first probe A | TTGGAGCT | (SEQ ID NO:3) |
| wild-type template | 3′-TATTTGAACACCATCAACCTCGACCA-5′ | (SEQ ID NO:1) |

Detection of a mutation in the second base in codon 12 may be performed by using the same second probe as above (second probe A), and a first probe, identified as first probe B below, that is complementary to a sequence terminating immediately adjacent (3′) to the second base of codon 12. Hybridization of probes suitable for detection of a mutation in the second base of codon 12 are shown below:

| | | |
|---|---|---|
| second probe A | 5′-ATAAACTTGTGGTAG | (SEQ ID NO:2) |
| first probe B | TGGAGCTG | (SEQ ID NO:4) |
| wild-type template | 3′-TATTTGAACACCATCAACCTCGACCA-5′ | (SEQ ID NO:1) |

Detection of a mutation at the third position in codon 12 is accomplished using the same second probe as above, and first probe C, which abuts the third base of codon 12. Hybridization of probes suitable for detection of a mutation in the third base of codon 12 are shown below

| | | |
|---|---|---|
| second probe A | 5′-ATAAACTTGTGGTAG | (SEQ ID NO:2) |
| first probe C | GGAGCTGG | (SEQ ID NO:6) |
| wild-type template | 3′-TATTTGAACACCATCAACCTCGACCA-5′ | (SEQ ID NO:1) |

In methods for detection of mutations at the second and third nucleotides of codon 12 described above, the second probe is 1 and 2 nucleotides, respectively, upstream of the region to which the first probe hybridizes. Alternatively, second probes for detection of the second and third nucleotides of codon 12 may directly abut (i.e., be exactly contiguous with) their respective first probes. For example, an alternative second probe for detection of a mutation in the third base of codon 12 in K-ras is:

5′-ATAAACTTGTGGTAGTT (SEQ ID NO: 5)

The detection of mutations can also be accomplished with a segmented primer comprising a series of at least three first probes. A series of first probes suitable for detection of a mutation in the third base of codon 12 is shown below:

| | | |
|---|---|---|
| first probe X | 5′-ATAAACTT | (SEQ ID NO:7) |
| first probe Y | TGGTAGTT | (SEQ ID NO:8) |
| first probe Z | GGAGCTGG | (SEQ ID NO:6) |
| wild-type template | 3′-TATTTGAACACCATCAACCTCGACCA-5′ | (SEQ ID NO:1) |

In a preferred embodiment multiple cycles of the single base extension reaction are performed using segmented primers comprising a donor molecule. First and second probes are exposed to sample under hybridization conditions that do not favor the hybridization of the short first probe in the absence of the longer second probe. Factors affecting hybridization are well known in the art and include raising the temperature, lowering the salt concentration, or raising the pH of the hybridization solution. Under unfavorable hybridization conditions (e.g., at a temperature 30-40° C. above first probe Tₘ), first probe forms an unstable hybrid when hybridized alone (i.e., not in the presence of a second probe) and will not prime the extension reaction. The longer, second probe, having a higher Tₘ, will form a stable hybrid with the template and, when hybridized to substantially contiguous portions of the nucleic acid, the second probe will impart stability to the shorter first probe, thereby forming a contiguous primer.

In a preferred embodiment, a modification of the dideoxy chain termination method as reported in Sanger, Proc. Nat'l Acad. Sci. (USA), 74:5463-5467 (1977), incorporated by reference herein, is then used to detect the presence of a mutation. The method involves using at least one of the four common 2′, 3′-dideoxy nucleotide triphosphates (ddATP, ddCTP, ddGTP, and ddTTP) comprising an acceptor molecule. A DNA polymerase, such as Sequenase™ (Perkin-Elmer), is also added to the sample mixture. In a preferred embodiment, a thermostable polymerase, such as Taq or Vent DNA polymerase is added to the sample mixture. Using the substantially contiguous first and second probes as a primer, the polymerase adds one ddNTP to the 3′ end of the first probe, the incorporated ddNTP being complementary to the nucleotide that exists at the single-base polymorphic site. Because the ddNTPs have no 3′ hydroxyl, further elongation of the hybridized probe will not occur. Chain termination will also result where there is no available complementary ddNTP (or deoxynucleotide triphosphates) in the extension mixture. After completion of the single base extension reaction, the donor and acceptors interact to produce a detectable signal.

Also in a preferred embodiment, deoxynucleotides, each comprising a donor molecule, may be used for detection if either the extension reaction is stopped after addition of only one nucleotide corresponding to the complement of the expected mutation, is exposed to the sample.

In the simplest embodiment of the invention, the nucleotide triphosphate mixture contains just the ddNTP (or dNTP) comprising an acceptor molecule that is complementary to the known mutation. For example, to interrogate a sample for a C→A mutation in the first nucleotide of codon 12 of the K-ras gene, second probe A and first probe A are exposed to an extension reaction mixture containing ddTTP (or dTTP). The incorporation of a ddTTP (or dTTP) in first probe A indicates the presence of a C→A mutation in the first nucleotide of codon 12 of the K-ras gene in the sample tested. First probe A co-hybridized with second probe A to a wild-type template will not be extended or, alternatively, will be extended with ddGTP (or dGTP) if available in the reaction mixture.

Given the large number of mutations that have been associated with colorectal cancer, a detection method for this disease preferably screens a sample for the presence of a large number of mutations simultaneously in the same reaction (e.g., apc, K-ras, p53, dcc, MSH2, and DRA). As described above, only very limited multiplexing is possible with detection methods of the prior art. Since methods of the present invention eliminate false positive signals resulting from the tolerance of mismatches of the longer second probes, the use of segmented oligonucleotide avoids the need for optimization of hybridization conditions for individual probes and permits extensive multiplexing. Several segmented primers can be assayed in the same reaction, as long as the hybridization conditions do not permit stable hybridization of short first probes in the absence of the corresponding longer second probes.

In a preferred embodiment, the primer extension reactions are conducted in four separate reaction mixtures, each having an aliquot of the biological sample, a polymerase, and the three complementary non-wild-type ddNTPs (or dNTP). Optionally, the reaction mixtures may also contain the complementary wild-type ddNTP (or dNTP). The segmented primers are multiplexed according to the wild-type template. In the present exemplification, the first two nucleotides coding for amino acid 12 of the K-ras gene are cysteines. Accordingly, second probe A and first probes A and B are added to a reaction mixture containing ddATP (or dATP), ddTTP (or dTTP), and ddCTP (or dCTP). Second probe C and first probe C are added to a reaction mixture containing labeled ddATP (or dATP), ddCTP (or dCTP), and ddGTP (or dGTP). Any incorporation of a ddNTP in a first probe indicates the presence of a mutation in codon 12 of the K-ras gene in the sample. This embodiment is especially useful for the interrogation of loci that have several possible mutations, such as codon 12 of K-ras.

In an alternative preferred embodiment, the primer extension reactions are conducted in four separate reaction mixtures, each containing only one complementary non-wild-type ddNTP or dNTP and, optionally, the other three unlabeled ddNTPs or dNTPs. Segmented primers can be thus be exposed only to the ddNTP or dNTP complementary to the known mutant nucleotide or, alternatively, to all three non-wild-type labeled ddNTPs or dNTPs. In the K-ras example provided above, if the first nucleotide of K-ras codon 12 is interrogated for a known C→G mutation, first probe A and second probe A are added to only one reaction mixture, the reaction mixture containing ddCTP (or dCTP). Optionally, methods of the invention may be practiced as described above using deoxynucleotides.

However, since several mutations have been identified at codon 12 of the K-ras gene, the probes are exposed to all non-wild-type ddNTPs or dNTPs. Thus, second probe A and first probes A and B are added to the three reaction mixtures containing ddATP (or dATP), ddTTP (or dTTP), or ddCTP (or dCTP). Second probe C and first probe C are added to the three reaction mixtures containing one of ddATP (or dATP), ddCTP (or dCTP), and ddGTP (or dGTP). Again, the extension of a first probe with a terminal nucleotide indicates the presence of a mutation in codon 12 of the K-ras gene in the biological sample tested.

In a preferred embodiment, several cycles of extension reactions are conducted in order to amplify the assay signal. Extension reactions are conducted in the presence of an excess of first and second probes, dNTPs or ddNTPs, and heat-stable polymerase. Once an extension reaction is completed, the first and second probes bound to target nucleic acids are dissociated by heating the reaction mixture above the melting temperature of the hybrids. The reaction mixture is then cooled below the melting temperature of the hybrids and first and second probes permitted to associate with target nucleic acids for another extension reaction. In a preferred embodiment, 10 to 50 cycles of extension reactions are conducted. In a most preferred embodiment, 30 cycles of extension reactions are conducted.

What is claimed is:

1. A method for identifying a nucleotide, the method comprising the steps of:
(a) exposing a biological sample to a nucleic acid primer capable of hybridizing with a nucleic acid and comprising a donor molecule;
(b) performing a primer extension reaction in the presence of a nucleotide complementary to the target nucleotide and comprising an acceptor molecule capable of interacting with said donor molecule to produce a detectable signal; and
(c) identifying the target nucleotide incorporated into said primer as a function of said signal.

2. The method of claim 1, wherein said donor activates said acceptor to produce a detectable signal.

3. The method of claim 2, wherein said signal is a photo-emitting signal.

4. The method of claim 1, wherein said extension reaction is performed in the presence of at least two different nucleotides, each comprising a different acceptor molecule.

5. The method of claim 1, wherein less than all the nucleotides complementary to the target nucleotide comprise an acceptor.

6. The method of claim 4 wherein each acceptor molecule produces a distinct signal.

7. The method of claim 1, wherein said signal is a fluorescent signal characteristic of the donor-acceptor interaction.

8. The method of claim 1, wherein said donor and acceptor molecules comprise a fluorophore.

9. The method of claim 1, wherein said donor and acceptor molecules comprise a fluorescent dye.

10. The method of claim 9, wherein said fluorescent dye is selected from the group consisting of 6-carboxyfluorescein (FAM), 6-carboxy-X-rhodamine (REG), N₁, N₁ N¹, N¹-tetramethyl-6-carboxyrhodamine (TAMARA), 6-carboxy-X-rhodomine (ROX), fluorescein, Cy5® or LightCycler-Red 640.

11. The method of claim 1 wherein said donor molecule further comprises 6-carboxyfluorescein (FAM).

12. The method of claim 11 wherein said acceptor molecule comprises), 6-carboxy-X-rhodomine (ROX).

13. The method of claim 1 wherein said nucleotide is a chain-terminating nucleotide.

14. The method of claim 13 wherein said chain-terminating nucleotide is a dideoxy nucleotide.

15. The method of claim 13 wherein said chain-terminating nucleotide is a 2′3′-dideoxy nucleotide triphosphates selected from the group consisting of ddATP, ddCTP, ddGTP, ddTTP and ddUTP.

16. The method of claim 1 wherein said nucleic acid is isolated from a biological sample selected from the group consisting of pus, semen, sputum, semen, saliva, cerebrospinal fluid, stool, urine, blood, biopsy tissue and lymph.

17. The method of claim 1 wherein said nucleic acid sample is obtained from stool.

18. The method of claim 1, wherein said target is a nucleic acid mutation.

19. The method of claim 15, wherein said mutation occurs in a gene selected from the group consisting of ras oncogenes, p53, dcc, apc, mcc and β-catenin.

20. A method for identifying a single nucleotide polymorphic variant, comprising the steps of:
exposing a sample to a first nucleic acid primer comprising a donor molecule, wherein said primer is capable of hybridizing to a nucleic acid in said sample at a locus immediately 5′ to a single nucleotide polymorphic locus;
extending said primer in the presence of at least two nucleotides, each comprising a different acceptor molecule capable of interacting with said donor molecule to produce a detectable signal;
detecting said signal; and
identifying said one or more nucleic acids present at said polymorphic locus.

21. The method of claim 20, wherein said nucleotides are chain-terminating nucleotides.

22. The method of claims 1 or 17, wherein said biological sample is obtained from a pooled patient population.

23. The method of claim 22 wherein said pooled biological sample comprises a stool sample obtained from members of a patient population.
